(19) United States
(12) Patent Application Publication
(10) Pub. No.: US 2006/0183165 A1
(43) Pub. Date: Aug. 17, 2006

(54) Combination of chemical differentiators and their applications in mass sensing-based chemical sensor systems

(76) Inventors: Sean Xiao-An Zhang, Cupertino, CA (US); Sui-Hing Leung, Cupertino, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/059,430

(22) Filed: Feb. 15, 2005

(51) Int. Cl. G01N 33/542; C12M 1/34; G01N 33/53

(52) U.S. Cl. 435/7.9; 435/287.2

(57) ABSTRACT

A sensing unit comprises a chemical sensing element and a transducer operatively associated therewith, wherein the chemical sensing element comprises at least one receptor for receiving a molecule of an analyte. An array of chemical sensing units is also provided, together with a chemical sensor system and a method of detecting and identifying the analyte.

### CROSS-REFERENCE TO RELATED APPLICATION

The present application is related to application Ser. No. ______, filed on even date herewith [PD-200312489-1], the contents of which are incorporated herein by reference. That application is directed to methods for fabricating the multi-sensor arrays with different chemical differentiators.

### TECHNICAL FIELD

The present invention is directed to chemical sensors, and, more particularly, to microscale and nanoscale chemical sensors, in which the critical dimension of the sensors is measured in micrometers or nanometers, respectively.

### BACKGROUND ART

Using mass-sensitive devices in microanalysis has been known for more than a century. In one example, piezoelectric resonance from a quartz crystal was used in a gravimetric microbalance, wherein a shift in resonance frequency accompanied an infinitesimal mass change in a mechanical oscillator. The existence of waves that are propagated only in relatively thin surface layers was also known, but it was not until relatively recently that these so-called surface acoustic waves (SAWs) were incorporated into a chemical gas sensor. The chief advantages of SAWs are increased sensitivity relative to BAWs (bulk acoustic waves) of lower resonant frequencies and a greater potential for miniaturization. On the other hand, SAWs require a relatively high operating frequency required for adequate sensor sensitivity.

A modification of this type of wave device uses a SAW sensor having an exceptionally thin membrane-like piezoelectric region in the substrate directly beneath the acoustic path. This in turn leads to the production of Lamb waves, which have proven to be particularly suitable for chemical sensor applications, opening the way to the so-called plate-mode oscillator, a device representing the present state of the art. Since then, numerous reports on chemical or biosensors technologies by combining both a mass sensitive device and sensing element have appeared in the literature.

Even though numerous sensory systems or products have been developed, however, all of these systems have limitations in terms of detecting capability. They only target a single, or at most a few, specific chemical or biological species. Furthermore, most of these devices are fairly large in size, which are not very convenient to carry (portability), and both their sensitivity and selectivity are usually not very high.

Thus, a highly selective and highly sensitive detecting system, capable of micro- and nano-detecting over a broad range, is needed.

### DISCLOSURE OF INVENTION

In accordance with the embodiments disclosed herein, a sensing element comprises a chemical sensing element and a transducer operatively associated therewith. The chemical sensing element comprises at least one receptor for receiving a molecule of an analyte.

Further in accordance with the embodiments disclosed herein, an array of chemical sensing elements is provided. Each chemical sensing unit comprises a chemical sensing element and a transducer. Each chemical sensing element comprises at least one receptor for receiving a molecule of an analyte.

Still further in accordance with the embodiments disclosed herein, a chemical sensor system is provided. The chemical sensor system comprises an array of chemical sensing units, each chemical sensing unit comprising a chemical sensing element and a transducer. Each chemical sensing element comprises at least one receptor for receiving a molecule of an analyte. The chemical sensor system is capable of identifying at least one molecule of an analyte.

Yet further in accordance with the embodiments disclosed herein, a method of detecting and identifying an analyte is provided. The method comprises:
providing a chemical sensor system comprising, in sequence, a sampling and pre-measurement compartment, a sample filtering and concentration compartment, at least one molecule sensing compartment, and a data processing unit;
introducing the analyte into each compartment in turn;
sensing a mass of the at least one molecular species;
sensing at least one chemical functional group on the at least one molecular species;
sensing a backbone structure of the at least one molecular species; and
analyzing information relating to the mass, the at least one functional group, and the backbone structure to provide an identity of the at least one molecular species of the analyte.

### BRIEF DESCRIPTION OF THE DRAWINGS

FIG. 1 is a schematic representation of a generalized sensing unit according to an embodiment herein;

FIG. 2 depicts an embodiment comprising a sensing unit in which a quartz crystal microbalance (QCM) is the transducer;

FIG. 3 depicts an example of a chemical sensor system based on a combination of chemical sensing units in an array, including molecular sensing compartments;

FIG. 4 depicts the molecule sensing compartments of FIG. 3 in greater detail; and

FIG. 5 depicts an embodiment of the sensor.

### BEST MODES FOR CARRYING OUT THE INVENTION

Reference is made now in detail to specific embodiments, which illustrates the best mode presently contemplated by the inventors for practicing the invention. Alternative embodiments are also briefly described as applicable.

In accordance with the teachings herein, a sensing unit comprises a chemical differentiator, or chemical sensing element, and a transducer operatively associated therewith. The chemical sensing element comprises at least one receptor for receiving a molecule of an analyte. Arrays of such sensing units may be constructed to detect and analyze an analyte and its components. As used herein, an analyte is a chemical substance that is undergoing analysis or is being measured so that its chemical composition and structure may be identified, and may be a single molecular species or a mixture of molecular species.

The technology disclosed herein is directed building micro- or nano-sensor arrays based on known mass-sensitive devices. The primary objective of the present teachings is to provide a unique combination of chemical differentiators in the sensor arrays and a method to use them for detecting the presence of a broad range of organic chemicals, biological specimens or toxins and/or explosive molecules (analytes).

The field of chemical sensors is one of the fastest growing areas both in research and commercial fields. The applications of chemical sensors include quality and process control, biomedical analysis, medical diagnostics, defense and national security, environmental pollution control, continuous and long term monitoring of pollutants, forensics, and fragrance analysis.

The need to identify multiple analytes simultaneously has led to the development of array-based sensors, disclosed herein. The design of a cross-reactive array generates a pattern upon exposure to an analyte mixture. This can be unique for a specific chemical compound and can be used to identify the chemical using some pattern-matching software. In order to develop a highly selective, highly sensitive, and universal sensor system, a micro- or nano-sensor array with multiple different sensing elements, each connected to its own specific transducer, has been regarded as one of the ultimate solutions possible. Sensor arrays offer several advantages over single sensor. It has better sensitivity to a wider range of analytes. It offers better selectivity, multi-component analysis, and analyte recognition rather than mere detection.

Sensor arrays are more analogous to human olfaction systems containing multiple receptors, whose responses are interpreted by neuron odor recognition processes. The posterior of the human nose has a region called the epithelium, which plays a major role in odor detection. This region has specialized neuron cells, which establish contact between brain and the outer world. Hair-like structures called cilia, which protrude from the neurons, extend out of each cell and have direct contact with odor. The other end of the cell is a fiber known as axon, which enters the brain. The odor molecule usually comes into contact with cilia that have a special protein for sensing purposes. This protein builds itself around the odor molecule and this sends an electrical signal to the olfactory bulb. The olfactory bulb acts as a relay station and transfers a signal to cortex of the brain where the interpretation of odor is done.

The basic principle of the technology disclosed herein is that a universal sensing system can be constructed by a combination of known technology of mass-sensitive devices (e.g., quartz crystal microbalance (QCM), etc.) and a certain array of chemical differentiators or receptors. While the related application indicated above (Ser. No. ______) describes a method for fabricating chemical sensor arrays in general, the present teachings relate to the integration of a certain array of selected chemical differentiators in conjunction with certain transducers to form a system capable of recognizing a broad spectrum of analytes.

The present teachings are believed to provide the first example of an artificial chemical nose to date that exhibits superior performance over any existing single chemical sensor detectors or other types of array sensors in terms of sensitivity and detection capability.

The unique combination of a universal mass sensing element with arrays of chemical functional group elements and molecular backbone sensing elements enable detection of a broad range of chemicals, biological toxins, and/or explosive materials conveniently and rather quickly.

The sensor device based on this technology functions as a mini-analytical lab in that it is simple, handy, extremely small in size, fast, highly sensitive, and extremely selective and yet its overall cost is very low.

The present teachings solve both problems of poor selectivity and narrow detection range that are usually associated with conventional chemical sensors.

The system disclosed herein provides sufficient information on three essential parameters used for the determination of the molecular identity of an analyte: (1) its unit mass or molecular weight, (2) the functional groups it comprises, and (3) its molecular structure. The system uses a universal mass-sensing unit to determine the molecular mass of the analyte. The system employs an array of well-selected chemical functional group sensing elements to differentiate the chemical functional group(s) of the analyte. The system utilizes array of carefully matched molecular backbone structure sensing elements to detect the possible molecular backbone structure of the unknown analyte.

The unique combination of the universal mass sensing and sensor arrays increases the selectivity and sensitivity greatly, and enables detection of a broad spectrum of analytes, more than any existing chemical sensor to date.

The chemical differentiator and its transducer function as a sensing unit, which in turn forms the basis of the chemical sensor system described below. FIG. 1 is a representation of a generalized sensing unit.

The sensing unit 10 comprises a chemical differentiator 12 and a transducer 14.

The chemical differentiator 12 is a chemical sensing element that interacts with the analyte environment and generates a physical response, which is not, in general, readily usable by the user or the system per se. A plurality of different chemical differentiators may be used in concert to provide various pieces of information about an analyte and its molecular makeup.

The second component of the sensing unit 10 is the transducer 14, which reads the response and converts it to another interpretable and quantifiable signal. The chemical differentiator, or sensing element, 12 is the sensor's heart and can be considered the primary part of the sensor, or sensing unit, 10. The nature, selectivity, and sensitivity of a chemical sensor 10 are highly dependent on the choice of the sensing element 12. As the sensing element 12 gets more complex, its sensing capability may be greatly improved.

The sensing element (receptor) 12 is an interface between the analyte environment, denoted 16, and the transducer 14. Usually, the sensing element 12 captures the analyte molecules in the gaseous state with a certain selectivity, which induces physical changes in the sensing element because of their chemical interaction. The physical variations are called “intermediate quantities”. The choice of a particular sensing element is based on the sensitivity, selectivity, reliability, and the reversibility of the related sensing mechanism, as is known.

The most important part in sensor development is the sensing element (receptor) 12 at which the molecular or ionic recognition process takes place, since this defines the overall selectivity of the entire sensor 10. The selectivity is the most important characteristic among all key parameters associated with a chemical or biological sensor, since it largely determines the trueness of the analytical method.

The analyte recognition process takes place either at the surface 12a of the sensing element 12 or in the bulk of the material, leading to a concentration-dependent change in some physical property that can be transformed into an electrical signal by the appropriate transducer. The transducer cannot itself improve the selectivity (exceptions are transducer-like electro-chemical electrodes which intrinsically deliver an analyte proportional signal), but it is responsible for the sensitivity of the sensor, and it functions together with the sensing element to establish a concentration dynamic range.

The electrical output signal of the transducer 14 is usually amplified by an electronic device (not shown) positioned close to or even integrated into it. If extensive electrical wiring is required between the sensor and the readout-control unit (not shown in FIG. 1), then the electrical signal might also be digitized as one way of minimizing noise pick-up during signal transmission. Several chemical responses produced by multiple sensing elements, each connected to its own transducer, can be transmitted via multiplexing techniques. In such a case, only a two-wire electrical connection to the control unit is needed as it also delivers the electrical power.

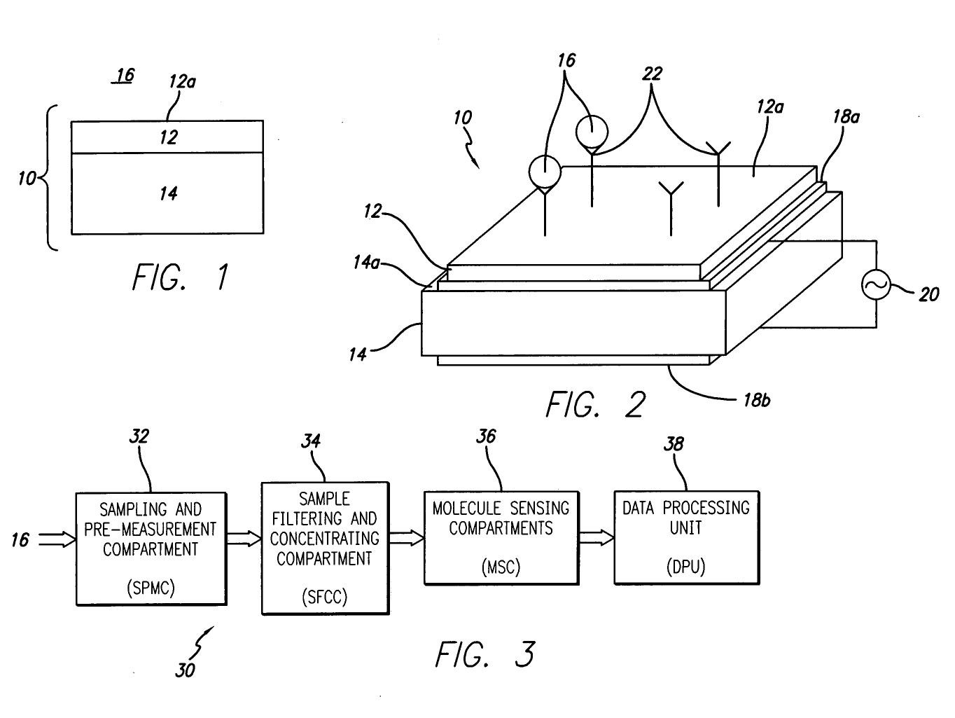

FIG. 2 depicts an embodiment of a sensing unit 10 in which a quartz crystal microbalance (QCM) is the transducer 14. In this embodiment, the chemical sensing element 12 is deposited on top (or both the top and the bottom) of one of the electrodes 18a, 18b of the quartz crystal microbalance, which is driven by oscillator 20 attached to the two electrodes. The chemical sensing element 12, which is typically a monolayer thick, comprises one or more receptors 22 that are uniquely adapted to receive some aspect (e.g., mass, functional group, structure) of the analyte molecule 16, as described in greater detail below.

The adsorption of molecules 16 increases the mass bound to the coated quartz crystal surface 14a and, under dry conditions, causes the crystal 14 to change its oscillation frequency according to the Sauerbrey equation:
Δf=−2.26×10⁻⁶f²(Δm/A)
where Δf in Hertz is the change in oscillation frequency of the quartz crystal, Δm in grams is the additional mass adsorbed onto crystal, A is the area of the coated quartz crystal in cm², and f is the resonance frequency of the quartz crystal in Hertz. For a given quartz oscillator in a given transducer, a change in the oscillation frequency is a function of the concentration or quantity of the analyte molecule 16 in the testing sample. Initial and periodic calibration may be needed to assure and maintain the accuracy of the QCM transducer in a sensing unit.

It will be appreciated that the chemical sensing element 12 may be deposited or formed directly on the surface of one or both electrodes 18a, 18b. Alternatively, either or both of the electrodes 18a, 18b may be chemically treated to function as a chemical sensing element 12. In this connection, reference is made to the related patent application cited above (Ser. No. ______).

As a minimum, three parameters must be known in order to determine the molecular identity of an analyte: (1) its unit mass or molecular weight, (2) the functional groups it comprises, and (3) its molecular structure. The chemical sensing elements for the sensing array are specifically chosen to produce information on these three molecular parameters. The sensing elements can be broadly classified into three categories. Universal Mass Sensing Units belong to the first category. Sensing elements in the second and third categories are respectively called the Chemical Functional Group Sensing Units and the Molecular Backbone Sensing Units.

Universal Mass Sensing Unit (UMSU).

The purpose of a Universal Mass Sensing Unit (UMSU) is to determine the molecular weight of the analyte 16. Since this sensing unit will work with all analytes under investigation, it is known as a Universal Mass Sensing Unit 10, and comprises a Universal Mass Sensing Element (UMSE) 12 and a transducer 14. The universal mass sensing element 12 employed in the UMSU is sometimes referred to herein as the universal adsorbent.

Solubility and adsorption characteristics are very important properties of the universal adsorbent layer. One of the desirable qualities in a universal adsorbent is its universal interaction with the sorbed material for achieving the desired sensitivity. Dipole-to-dipole and hydrogen bonding interactions are examples of orientation dependent solubility interactions.

In one embodiment, the universal mass sensing unit's transducer is a quartz crystal microbalance (QCM), although other types of transducers may be deployed.

The universal adsorbent can be any organic small molecular or polymeric material that is capable of adsorbing a broad range of chemicals or biological molecules. The adsorbent can also be a layer of activated carbon, silica gel, aluminum oxide gel or other type of inorganic material.

There are three aspects for an ideal universal adsorbent: (1) excellent adsorption for a broad range of analytes, (2) excellent reversion, and (3) good repeatability (identical molar absorption capability for different molecules or analytes).

With regard to the first aspect of being an excellent adsorbent, the universal adsorbent should be capable of adsorbing a broad range of organic or biological molecules quickly through physical forces (e.g., hydrogen bonding, van der Waals interaction, etc.) under certain conditions (e.g., room temperature or atmospheric pressure, etc.).

With regard to the reversion aspect (2), the universal adsorbent should be able to release those prior-adsorbed molecules completely during a thermal desorption reactivation process. This property is very important to ensure that the sensor can be re-used a number of times (so at least calibration is possible) and have a long lifetime.

With regard to the good repeatability aspect (3), the universal adsorbent should be able to adsorb the same molar quantity of molecules each time. The repeatability is an extremely important property for identifying the molecular weight of the unknown molecule (analyte). The maximum unit molar adsorption for this type of mass sensing device is determined through a calibration process with a known molecular material (a standard sample). Based on the unit molar adsorption information of the adsorbent, one can determine not only how many unknown molecules the device can fully adsorb, but also what is the molecular mass of this unknown molecule (analyte) by measuring directly the mass of the analyte. With regard to identical molar absorption capability for different molecules or analytes, this is a useful criterion for the UMSU to function. In order to arrive at the molecular weight of the analyte, the sensing element has to measure the same molar quantity of the analyte every time it measures the weight of the analyte even though the exact molar quantity may not be known.

A series of calibration runs with several known chemical compounds of known molecular weights has to be taken beforehand, from which it is possible to construct a graph of the transducer's response (delta frequency) as a function of molecular weight for the given UMSU of a particular molar absorption capacity. As noted in the Sauerbrey equation above, one may conclude that the delta frequency varies monotonically with molecular weight, since the same molar quantity is being measured, whether it be the analyte or the calibration compound. Because of this monotonicity, one can deduce the molecular weight of the unknown analyte as soon as one knows the delta frequency reading from the transducer.

Many commercial available materials in the chromatography industry can be used as a universal adsorbent with or without chemical modification (e.g., C-8 or C-18 coating for GC or LC chromatography, etc.)

The universal adsorbent can be deposited onto the surface of mass sensing transducer via either a chemical bonding (e.g., covalent bond) or physical adsorption (e.g., hydrogen bonding, van der Waals interaction, etc.). Covalent bonding is usually the preferred way to link the universal adsorbent onto the transducer.

Potential candidates for the universal mass-sensing element employed in the UMSU are shown in Table I, below.

TABLE I
Universal Mass Sensing Element (U-1 to U-6)

| # | Universal Adsorbent | Targets |
|---|---|---|
| U-1 | Coating materials for GC or LC | Most organic materials |
| | (e.g., C-8 or C-18, etc.) | |
| U-2 | Activated carbon | Most organic materials, |
| | (e.g., poly-aromatic materials) | especially good at aromatics |
| U-3 | Silica gel | Most organic materials, |
| | (e.g., materials used for | especially good at |
| | chromatography, etc.) | polar organic materials |
| U-4 | Aluminum gel | Most organic materials, |
| | (e.g., materials used for | especially good at polar |
| | chromatography, etc.) | organic materials |
| U-5 | Organometallics | Most organic materials, |
| | (e.g., metal phthalocyanine or its | especially good at aromatics |
| | derivatives, etc.) | |
| U-6 | Organic materials | Most organic materials |
| | (e.g., substituted cyclodextrin, etc.) | |

B. Chemical Function Group Sensing Units (CFGSU).

Sensing elements in the second category are dedicated to the detection of the constituent functional group(s) in an analyte. Different types of functional group sensing elements, in general, can be one or more of the following: a strong acid (SA), a strong base (SB), a weak acid (WA), a weak base (WB), a hydrogen bonding accepting group (HBAG), a hydrophilic group (HIG), an electron donor (ED), an electron acceptor (EA), etc. Each CFGSU 10 comprises a Chemical Function Group Sensing Element (CFGSE) 12 and a transducer 14.

The SA or strong acid type of differentiator (or group) preferentially binds with any basic group of the analyte (both strong base and weak base) to form a proton-exchanged ionic complex. A specific example is the difluoro-acetic acid (—CF₂CO₂H) group, which can detect a range of basic groups (both strong acid and weak base) by forming an inter-molecular ionic complex with them. The strong base functional group can be, but is not limited to, any one of the following: —RSO₃H (e.g., —C₆H₄SO₃H, etc.), —CO₂H (with or without a strong electron withdrawing group to activate the acid group (e.g., —CF₂CO₂H, etc.)), —PO(OH)₂, and —PO(OR)OH, etc. The —CF₂CO₂H group is chosen here to illustrate the present teachings.

The SB or strong base type of differentiator (or group) preferentially binds with any acidic group of the analyte (both strong acid and weak acid) to form a proton-exchanged ionic complex. A specific example is an N,N-dimethyl alkyl amine (—(CH₂)ₙ—N(CH₃)₂) group, which can detect a range of acidic groups (both strong acid and weak acid) by forming an inter-molecular ionic complex with them. The strong base functional group can be, but is not limited to, any one of the following: alkyl amines (including the primary amines, secondary amines, and tertiary amines), imines, guanidine, hydrazine and/or its derivatives, azo and/or its derivatives, hydroxylamine and/or its derivatives, alkyl phosphines (including primary, secondary, and tertiary phosphines), alkyl aryl phosphines (including dialkyl aryl phosphines, and alkyl diaryl phosphines, etc.), heterocycles containing N or P (e.g., pyridine and/or its derivatives, imidazole and/or its derivatives, etc.).

The WA or weak acid type of differentiator (or group) preferentially binds only with a strong basic group of the analyte to form a proton-exchanged ionic complex. A specific example is a phenol (—C₆H₅—OH) group, which can detect a range of strong basic groups by forming an inter-molecular ionic complex with them. The weak acid functional group can be, but is not limited to, any one of the following: —OH (phenol or other type of aromatic —OH group), aromatic —COOH (with strong electron donating group to deactivated the acid), —B(OH)₂ and its derivatives, —PO(OH)₂ (with strong electron donating group to deactivated the acid), etc.

The WB or weak base type of differentiator (or group) preferentially binds only with a strong acidic group of the analyte to form a proton-exchanged ionic complex. A specific example is an N,N-dimethyl aniline (—C₆H₅—N(CH₃)₂) group, which can detect a range of strong acidic groups by forming an inter-molecular ionic complex with them. The strong base functional group can be, but is not limited to, any one of the following: aromatic amines (including primary amines, secondary amines, and tertiary amines), deactivated imines (with a strong electron withdrawing group to deactivate the imine group), aromatic phosphines (e.g., secondary phosphines, such as biphenyl phosphine, and tertiary phosphines, such as triphenyl phosphine, etc.), deactivated aromatic phosphines (with one or more strong electron withdrawing groups on the aryl group to deactivate the aromatic phosphine group), etc.

The HBAG or hydrogen bond accepting type of differentiator (or group) preferentially binds with a hydrophilic group of the analyte to form a hydrogen bond. A specific example is a —CO—NR₁R₂ group, which can detect a range of hydrophilic groups by forming an inter-molecular hydrogen-bonding complex with them. The hydrogen bonding accepting group can be, but is not limited to, any one of the following: —CN, —NO₂, —C(═O)NH₂, —C(═O)NHR, —C(═NH)NH₂, —C(═NR)NH₂, —C(═NH)NHR, —C(═S)NH₂, —C(═O)—SH, —C(═NH)—SH, >C═O, —N═N—, —CN, —NO₂, —CF₃, —CCl₃, —CH═O, —SO—, SO₂—, —SO₂OR, —SO₂NR—, —COOR, —C(═O)NR—, and heterocycles containing nitrogen (e.g., pyrrole and/or its derivatives), oxygen (e.g., furan and/or its derivatives), or sulfur (e.g., thiophene and/or its derivatives), etc.

The HIG or hydrophilic type of differentiator (or group) preferentially binds with a hydrophilic group or hydrogen bond accepting group of the analyte to form hydrogen bonding. A specific example is a —OH group, which can detect a range of polar hydrogen bond accepting groups by forming an inter-molecular hydrogen-bonding complex with them. The hydrophilic group can be, but is not limited to, any one of the following: —OH, —NH₂, SH, —COOH, —C(═O)NH₂, —C(═O)NHR, —C(═S)NH₂, —C(═O)—SH, —C(═NH)NH₂, —C(═NR)NH₂, —C(═NH)NHR, —C(═NH)—SH, etc.

The electron donor or ED and the electron acceptor EA groups are further described in the Molecular Backbone Sensing Elements section below since they exhibit dual functionality, as both a CFGSE and a MBSE.

The electron acceptor or EA type of differentiator (or group) preferentially binds only with electron donor (a highly electron rich ring system) or hydrophilic group of the analyte to form an electron exchanged charge complex, a dipole complex or a hydrogen-bonding complex. A specific example is 7,7,8,8-tetra-cyanoquino-dimethane (TCNQ) group, which can detect a range of electron-rich nitrogen-, sulfur-, and oxygen-containing heterocycle ring systems. The electron acceptor functional group can be, but is not limited to, any one of the following: functional groups or structures containing highly electron negative heteroatoms or atomic groups (e.g., >C═O, —N═N—, —CN, —NO₂, —CF₃, —CCl₃, —CH═O, —SO—, SO₂—, —SO₂OR, —SO₂NR—, —COOR, —C(═O)NR—, —O—, F, Cl, Br, I, etc.

C. Array-Based Sensors.

Array-based sensors use pattern recognition techniques for analyte detection and this pattern recognition technique works on the basis of the quality and quantity of the information given to it in the form of numerical responses. Hence, the accuracy of the results is highly dependent on this numerical information supplied. Thus, the logical selection of sensing elements for the sensor array is very important, since different sensing elements will provide unique information for identifying different analytes Therefore, by having sensors for the full range of adsorbing interactions, detecting a particular analyte is possible with high accuracy via a pattern recognition algorithm.

Of the nine chemical functional group differentiators shown above, six types (SA, AB, WA, WB, HBAG and HIG) are now selected and illustrated in an embodiment below. However, other selections of chemical functional group differentiators are possible; such selections depend on the need of specific applications. The specific oriented interactions between the six selected functional group sensing elements and sorbed material(s) are summarized in Table II, below. The sensing elements for the six general functional groups, with some specific examples included in square brackets, are listed in the second column of Table II. A list of the target functional groups being detected is also given in the third column, as well as their unit or molecular mass in the fourth column.

TABLE II
Chemical Function Group Sensing Elements (F-1 to F-6)

| | | | | Unit (or | |
|---|---|---|---|---|---|
| | Sensing Element | Target Functional | Specific Examples of Functional | Molecular) | |
| # | [Specific Example] | Group | Groups Being Detected | Mass | Notes |
| F-1 | Stronger acid (SA) | Stronger and | —NH—NR₁R₂ | 31 + 14n | n (the number of |
| | [—CF₂CO₂H] | weaker base | —NR₁R₂ | 16 + 14n | carbon atoms in each |
| | | | —NR₁R₂ | 92 + 14n | molecular functional |
| | | | —NH(C═NH)NR₁R₂ | 58 + 14n | group) = 0, 1, 2, 3, . . . |
| | | | pyridine or its alkyl substituents | 79 + 14n | R₁ = H or alkyl |
| | | | amino-pyridine or its substituents | 94 + 14n | R₂ = H or alkyl |
| | | | imidazole or its alkyl substituents | 68 + 14n | |
| | | | pyrrole or its alkyl substituents | 67 + 14n | |
| F-2 | Weaker acid (WA) | Stronger base or | —NH—NR₁R₂ | 31 + 14n | n (the number of |
| | [φ-OH] | hydrogen bonding | —NR₁R₂ | 16 + 14n | carbon atoms in each |
| | | accepting group | —NH(C═NH)NR₁R₂ | 58 + 14n | molecular functional |
| | | | imidazole or its alkyl substituents | 68 + 14n | group) = 0, 1, 2, 3, . . . |
| | | | amino-pyridine or its substituents | 94 + 14n | R₁ = H or alkyl |
| | | | —CN | 26 | R₂ = H or alkyl |
| | | | —C(═O)R₁ | 29 + 14n | |
| | | | —NO₂ | 46 | |
| | | | —C(═O)NHR₁ | 44 + 14n | |
| F-3 | Stronger base (SB) | Stronger and | —CO₂H | 45 | n (the number of |
| | [-Alkyl-NR₁R₂] | weaker acid | —PO₃H₂ | 81 | carbon atoms in each |
| | | | —B(OH)₂ | 45 | molecular functional |
| | | | φ-OH | 93 + 14n | group) = 0, 1, 2, 3, . . . |
| | | | φ-SH | 109 + 14n | |
| F-4 | Weaker base (WB) | Stronger acid or | —CO₂H | 45 | |
| | [-φ-NH₂] | hydrogen bonding | —PO₃H₂ | 81 | |
| | | accepting group | —CN | 26 | |
| | | | —C(═O)R₁ | 29 + 14n | |
| | | | —NO₂ | 46 | |
| | | | —C(═O)NHR₁ | 44 + 14n | |
| F-5 | Hydrogen bonding | Hydrophilic group | —OH | 17 | R₁ = H or alkyl |
| | accepting group | | —SH | 33 | R₂ = H or alkyl |
| | (HBAG) | | —NH₂ | 16 | n [the number of |
| | [—CO—NR₁R₂] | | —NH(C═NH)NR₁R₂ | 58 + 14n | carbon atoms in the |
| | | | pyrrole or its alkyl substituents | 67 + 14n | alkyl group(s)] = 0, 1, |
| | | | imidazole or its alkyl substituents | 68 + 14n | 2, 3, . . . |
| F-6 | Hydrophilic group | Hydrogen bonding | —NH—(C═NH)—NR₁R₂ | 58 + 14n | n [the number of |
| | (HIG) | accepting group | —CN | 26 | carbon atoms in the |
| | [—OH] | | —C(═O)R₁ | 29 + 14n | alkyl group(s)] = 0, 1, |
| | | | —NO₂ | 46 | 2, 3, . . . |
| | | | —C(═O)NHR₁ | 44 + 14n | R₁ = H or alkyl |

Note:
φ = phenyl (—C₆H₅)

It can be seen from the Table II that each specific sensing element can detect a range of molecular functional groups based on their specific oriented chemical or physical interactions. However, different functional groups will usually interact with a unique sub-set of the six sensing elements in the sensor array. This characteristic signature, when combined with the knowledge of its molecular backbone and molecular mass, enables identification of the analyte in most cases.

D. Molecular Backbone Sensing Units (MBSU).

The backbone detectors can consist of one or more of the following: an electron donor (ED), an electron acceptor (EA), an aliphatic group (R), an aromatic group (Ar), a hydrophobic group (HOG), etc. As the name clearly implies, the sole purpose of the molecular backbone detectors in the array sensor is to identify the backbone structure of the analyte, which leads to the identity of the analyte itself. Each MBSU 10 comprises a Molecular Backbone Sensing Element (MBSE) 12 and a transducer 14.

The ED or electron donor type of differentiator (or group) preferentially binds only with an electron acceptor (EA) or a highly electron-deficient ring system of the analyte to form an electron-exchanged charge complex or a dipole complex. A specific example is the tetrathiafulvalene (TTF) group, which can detect a range of highly explosive, electron-deficient multi-nitro-aromatics or multi-cyano-aromatics (e.g., TNT (trinitrotoluene), etc). The electron donor functional group can be, but is not limited to, any one of the following: functional groups or structures containing electron-rich hetero atoms or atomic groups (e.g., O, S, N, P, B, Se, As, —OH, —SH, —NHR, —NR₂, —NH₂, etc.), or containing electron-rich unsaturated hydrocarbons (e.g., alkenes, alkyls, etc.).

The EA or electron acceptor type of differentiator (or group) preferentially binds only with an electron donor or a highly electron-rich ring system or hydrophilic group of the analyte to form an electron-exchanged charge complex, a dipole complex or a hydrogen-bonding complex. A specific example is the 7,7,8,8-tetra-cyanoquino-dimethane (TCNQ) group, which can detect a range of electron-rich nitrogen-, sulfur-, and oxygen-containing heterocycles ring system. The electron acceptor functional group can be, but is not limited to, any one of the following: functional groups or structures containing highly electron negative heteroatoms or atomic groups (e.g., >C═O, —N═N—, —CN, —NO₂, —CF₃, —CCl₃, —CH═O, —SO—, SO₂—, —SO₂OR, —SO₂NR—, —COOR, —C(═O)NR—, —O—, F, Cl, Br, I, etc).

The aliphatic group or R type of backbone structure detector preferentially binds with the same or different kind of aliphatic group in the analyte to form a molecular complex via van der Waals force interaction. A specific example is a 2-methyl-butane (—CH₂CH₂CH(CH₃)₂) group, which can detect a range of aliphatic chain backbone structures by forming an inter-molecular complex with them. The R type of backbone structure detector can be, but is not limited to, any one of the following: saturated hydrocarbons, unsaturated hydrocarbons, heteroatom-substituted hydrocarbons (including a linear, branched or cyclic system, etc.

The aromatic group or Ar type of backbone structure detector preferentially binds with an aromatic molecular structure in the analyte to form a molecular complex via van der Waals force interaction. A specific example is a pyrene (—C₁₆H₉) group, which can detect a range of aromatic ring systems by forming an inter-molecular complex with them. The Ar type of backbone structure detector can be, but is not limited to, any one of the following: single ring or multi-ring aromatic systems, aromatic heterocycles, heteroatom-substituted hydrocarbons, etc.

The HOG type of hydrophobic group preferentially binds with the same or different kinds of hydrophobic groups to form a molecular complex via van der Waals force interaction. The hydrophobic group can be, but is not limited to, any one of the following: aliphatic groups (including both saturated and unsaturated hydrocarbons), aromatic groups, ethers, thio ethers, esters, thiol esters, tertiary amines, etc.

E. Backbone Structure Sensing Elements.

Backbone structure sensing elements are used to uncover the molecular structure or backbone of the analyte. Four backbone sensing elements B-1 through B-4 as shown in Table III are identified as comprising one embodiment. Note that B-1, the Electron Donor, and B-2, the Electron Acceptor, could also act as functional group sensing elements. The combination of the four sensing elements B-1 through B-4 would be sufficient to detect most backbone structures in the analytes of interest.

TABLE III
Molecular Backbone Structure Sensing Elements (B-1 to B-4)

| | | | | Unit (or Mo- | |
|---|---|---|---|---|---|
| | Sensing Element | Target Backbone | Specific Examples of the Back- | lecular) | |
| # | [Specific Example] | Structure | bone Structure Being Detected | Mass | Notes |
| B-1 | 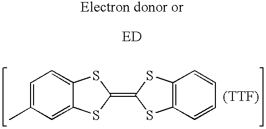 | Electron acceptor or a highly elec- tron deficient ring system | TCNQ Di-nitrobenzene or its substituted Tri-nitrobenzene or its substi- tuted Tri-nitro phenol or its alkyl substi- tuted | 180 + 14n 168 + 14n 213 + 14n 229 + 14n | n[the number of carbon atom in the alkyl group(s)] = 0, 1, 2, 3, . . . |
| B-2 | 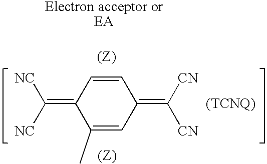 | Electron donor (highly electron rich ring system) or Hydrophilic group | Pyrene or its alkyl substituted Perylene or its alkyl substituted Thiophene or its alkyl substituted Pyrrole or its alkyl substituted Furan or its alkyl substituted Amino-pyridine or its substitute Pyridine or its alkyl substitute —OH —SH —NH₂ | 202 + 14n 252 + | |
| # 14n 80 + 14n 67 + 14n 68 + 14n 94 + 14n 79 + 14n 17 33 16 | n[the number of carbon atom in the alkyl group(s)] = 0, 1, 2, 3, . . . | | | | |
| B-3 | 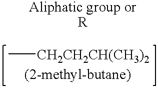 | Aliphatic back- bone | Alkanes [CₙH_{(2n + 2)}]Alkanes [CₙH_{(2n + 2 - 2m)}]Alkanes [CₙH_{(2n + 2 - 4m)}] | 14n + 2 14n + 2 − 2m 14n + 2 − 4m | n (the number of carbon atom in each molecule) = 1, 2, . . . m (the number of al- kene or alkyne in each mole- cule) = 1, 2, . . . |
| B-4 | 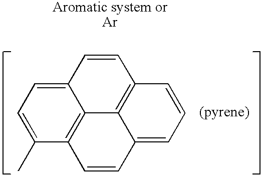 | Aromatic back- bone | 1-D structure [C_{(4m + 2) r}H_{(2m + 4)r}]2-d pyramid structure [C_{(2m + 1 + 3n)}H_{3(n + 1)}]{note: m = n(n + 1)/2}Thiophene or its alkyl substituted Pyrrole or its alkyl substituted Furan or its alkyl substituted Amino-pyridine or its substitute Pyridine or its alkyl | | |
| # substitute | (50m + 28)r (24m + 29n +15) 80 + 14n 67 + 14n 68 + 14n 94 + 14n 79 + 14n | m (number of fused ring in each aromatic unit) = 1, 2, . . . r (the number of aromatic unit in a molecule) = 1, 2, . . . n (the number of the levels of pyramid structure | | | |

Sensing elements in the second column of Table III comprise a set of general molecular backbone structure detectors with some specific examples cited in square brackets. A list of target backbone structures and functional groups for each sensing element is also given in the third column, together with the unit mass of the backbone structures and functional groups in the fourth column.

It can be seen from Table III that each specific sensing element can detect a range of molecular backbone structures and functional groups based on their specific oriented chemical or physical interactions. However, the molecular backbone structure or functional group of an analyte will usually interact with more than one type of the sensing element in the sensor array. This unique property of the sensing elements will help to distinguish one backbone from the others, making it possible to identify the molecular structure with the additional information of molecular mass.

F. A Chemical Sensor System.

In the following sections, a chemical sensor system, based on a novel combination of chemical sensing units 10 in an array, is described. FIG. 3 is a schematic overview of such a system 30.

The sensor system 30 described herein comprises four components: a sampling and pre-measurement compartment (SPMC) 32, a sample filtering and concentration compartment (SFCC) 34, molecule sensing compartments (MSC) 36, and a data processing unit (DPU) 38.

The sampling and pre-measurement compartment (SPMC) 32 is a front gate of the sensor system 30. It takes the sample (analyte 16) from the outside source and prepares it for further treatment in the next compartment (SFCC) 34. Typical preparatory task includes such process as pre-ionization, gasification, etc.

The sample filtration and concentration compartment (SFCC) 34 is a second stage sample preparation unit. It prescreens the sensing sample and removes any unwanted solvent(s) or water through filtering and concentration processes. The higher concentration of the analyte resulting from this compartment invariably yields a more reliable and accurate result.

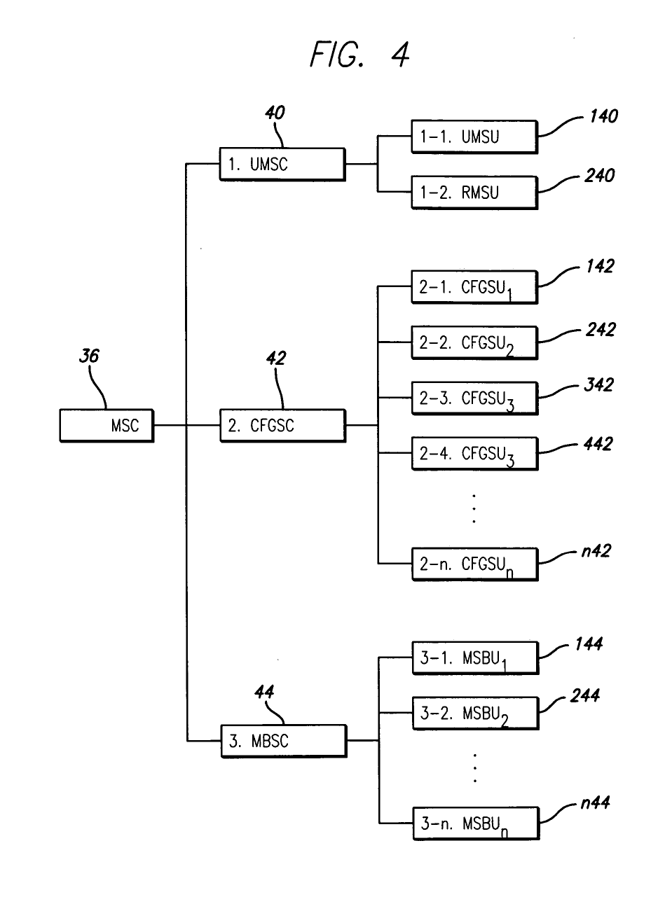

The molecule sensing compartments (MSC) 36 as depicted in FIG. 4 are the heart of the sensor system 30. The MSC 36 shown therein further comprises three sub-compartments: (1) a universal mass sensing sub-compartment (UMSC) 40, a chemical functional group sensing sub-compartment (CFGSC) 42, and a molecular backbone structure sensing sub-compartment (MBSC) 44. Each of these sensing compartments 40, 42, 44 is made up of sensing units as described below.

The Data Processing Unit (DPU) 38 acts as a main interface between the sensing units 10 and the user. The DPU first provides the requisite driving signals to the transducers 14 for them to operate. In return, raw signals from the sensing units 10, which are not readily usable to the user, will be passed to the DPU. They have to be processed, interpreted and properly formatted before being presented to the user. These are the main functions of the DPU.

Some of the DPU's functions may be carried out in software or firmware while others are implemented in electronic circuitry, which may be external to the system or integrated with the rest of system should it be fabricated on silicon or other solid state material.

Several steps that a user would have to follow in order to identify the analyte will be described in a later section. Nonetheless, to make the system more user friendly, these steps may be reduced to certain pre-defined algorithms as a system software or firmware resident in the DPU 38.

F1. Universal Mass Sensing Compartment.

With continuing reference to FIG. 4, the universal mass sensing sub-compartment 40 comprises a pair of mass sensing units: (1) a universal mass sensing unit (UMSU) 140 and (2) a reference mass sensing unit (RMSU) 240. The RMSU 240 is a special mass sensing unit without any sensing element on top of its transducer 14. The purpose of using a RMSU 240 here is to help minimize the system errors caused by changes in the external environment or to compensate for drift as the transducers age.

F2. Chemical Functional Group and Molecular Backbone Structure Sensing Compartments.

With continuing reference to FIG. 4, the chemical functional group sensing sub-compartment (CFGSC) 42 and the molecular backbone structure sensing sub-compartment (MBSC) 44 each comprise an arrays of chemical functional group sensing units (CFGSU) 142, 242, 342, 442, . . . n42 and molecular backbone structure sensing units (MBSU) 144, 244, . . . n44, respectively.

The CFGSUs 142, 242, 342, 442, . . . n42 are constructed from a select set of functional group sensing elements 12 such as listed in Table II and an equal number of individual transducers 14. Similarly, the MBSU 144, 244, . . . n44 is made up of an array of molecular backbone sensing units 10 employing sensing elements 12 such as listed in Table III and, again, an equal number of individual transducers 14. Though the CFGSC 42 and MBSC 44 serve different functions, nonetheless they are constructed in identical manner as fully disclosed in the above-referenced related application.

The following is a brief summary of the above-referenced related application (Ser. No. ______), in which several methods to make chemical sensor array are explained. To summarize, the basic idea is that selective sensing elements can be introduced onto different specific transducers (whether through chemical bonding or physical adsorption) by selectively activating the particular transducer (electrode) in an array while deactivating the remaining transducers. The activation is achieved by manipulating the electric field among the array of the transducers (electrodes). The sensing elements are designed with an ionizable connecting group. The ionizable connecting group can be a single heteroatom (e.g., —S—, —O—, etc.) or an atomic group (e.g., —NH—, —ONH—, —NR—, —NHNR—, —ONR—, —Si(OR)₂—, —O—Si(OR)₂—, —SiR₂—, —OSiR₂—, etc.). Each particular type of sensing element is pre-ionized, and is then introduced into the system containing one of more selectively activated micro- or nano-transducer(s) in the array. Methods for pre-ionization include electro-spraying and chemical ionization. Electro-spraying especially has been often used in the manufacture of mass spectrometers.

The Coulomb interaction between the pre-ionized sensing elements and the electric field (E-field) in the array can very precisely direct and highly selectively control the ion deposition process. The positive potential on the activated electrode attracts the negatively charged sensing elements onto its (or their) surface and promotes a chemical bonding reaction between them. The negative potential on those deactivated transducers (electrode) in the array structure repels the anionic sensing elements away from their surface and protects them from unwanted deposition. Through this type of sequential and selective activation and deposition process, a micro- or nano-sensor array with multiple different sensing elements, each connected to its own specific transducer, can be achieved.

G. An Embodiment.

When analytes to be detected are in trace concentrations and the environment includes interfering substances, the detection becomes complex, and usually array-based detectors are used under these circumstances. In such cases, each sensor of the array should be carefully chosen to obtain unique information on targeted analytes as well as information on potential interfering substances for known and unknown analytes. Arrays of CFGSUs and MBSUs, as opposed to a single sensing unit, provide much more useful information on different analytes because of different adsorbing interactions. Judicious selection of sensing elements produces sufficient information in most cases, leading to the identification of the analyte under investigation. Physical properties of a chemical functional differentiator or a molecular backbone detector should be decided carefully so as to maximize the sensor's selectivity by varying the adsorbing properties.

An understanding of the sorption and selectivity is important in the design of such sensing elements. Tailoring the physical and chemical properties of the sensing elements used in the sensors can control the selectivity and sensitivity of each sensor. It is well known in chemistry that weak interactions between vapor and sensor coating results with good reversibility and little hysterisis. But such weak interactions may not have sufficient sensitivity and selectivity. On the other hand, strong interactions may improve sensitivity and selectivity but result in sensors of irreversibility or very slow reversibility. Thus, if reversibility is important, a balance must be struck between selectivity and reversibility.

All three categories of sensing elements (universal mass sensing elements 40, chemical functional group sensing elements 42, and molecular backbone sensing elements 44) are selected based on an overall consideration of their desired selectivity, sensitivity, and the reversibility, and their specific oriented interactions between the sorbed material 16 and sensing elements. The molecular functional groups or backbone structures of the molecule preferentially bind with other type-specific functional groups or structures through certain chemical or physical interactions (e.g., the sorption of a vapor, which has a hydrogen bond base). The incorporation of hydrogen bond acidic groups causes strong and more selective interactions. Understanding of solute and solvent molecules solubility properties and their interactions will make a better sensor coating material. The specific oriented chemical or physical interaction between the sorbed material and sensing elements include, but is not limited to, proton exchange, charge transfer, Coulomb force, van der Waals force, hydrogen bonding, dipole interaction, etc.

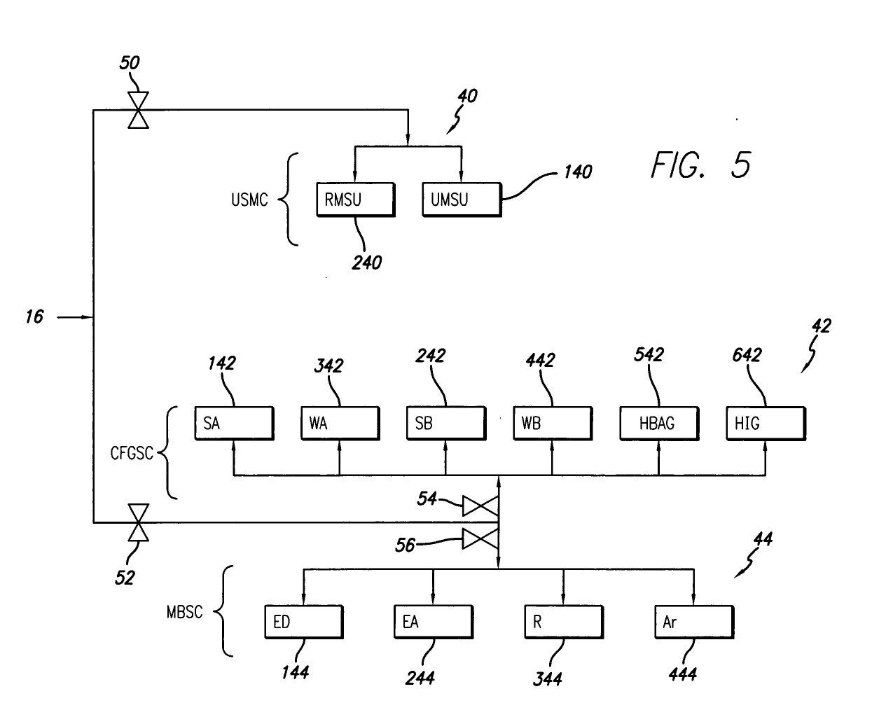

FIG. 5 depicts one embodiment, wherein the analyte 16 is introduced into the molecular sensing compartments section. Valve 50 controls flow of the analyte to the UMSC 40, while valve 52 controls flow of the analyte to both the CFGSC 42 and MBSC 44. Valves 54 and 56 control flow of the analyte to the CFGSC 42 and to the MBSC 44, respectively.

The UMSC 40 has been described in connection with FIG. 4, and is the same in this embodiment, namely, a pair of universal mass sensing units 140 and 240. The SFGSC 42 and MBSC 44 are now described in terms of the embodiment depicted in FIG. 5.

G1. Chemical Functional Group Sensing Elements—An Embodiment.

Six types of chemical functional group sensing elements (SA, SB, WA, WB, HBAG, and HIG) 142, 242, 342, 442, 542, 642 are selected and illustrated in FIG. 5. However, other combinations with more or less or different chemical functional group sensing elements can be selected, which depends on the needs of specific applications.

The SA or strong acid type of sensing element (or differentiator) 142 preferentially binds with any basic group in the analyte 16 (both strong base and weak base) to form a proton-exchanged ionic complex. A difluoro-acetic acid group [—CF₂CO₂H] is chosen here to illustrate this aspect.

The SB or strong base type of sensing element (or differentiator) 242 preferentially binds with any acidic group in the analyte 16 (both strong acid and weak acid) to form a proton-exchanged ionic complex. An N,N-dimethyl alkyl amine [—(CH₂)ₙ—N(CH₃)₂] group is chosen here to illustrate this aspect.

The WA or weak acid type of sensing element (or differentiator) 342 preferentially binds only with strong basic group in the analyte 16 to form a proton-exchanged ionic complex. A phenol group [—C₆H₅—OH] is chosen here to illustrate this aspect.

The WB or weak base type of sensing element (or differentiator) 442 preferentially binds only with strong acidic group in the analyte 16 to form a proton-exchanged ionic complex. An N,N-dimethyl aniline [—C₆H₅—N(CH₃)₂] group is chosen here to illustrate this aspect.

The HBAG or hydrogen bond accepting type of sensing element (or differentiator) 542 preferentially binds with a hydrogen bond providing group or hydrophilic group in the analyte 16 to form hydrogen bonding. An N,N-dialkyl amide [—CO—NR₁R₂] group is chosen here to illustrate this aspect.

The HIG or hydrophilic type of sensing element (or differentiator) 642 preferentially binds with a hydrophilic group or hydrogen bond accepting group in the analyte 16 to form hydrogen bonding. A hydroxyl [—OH] group is chosen here to illustrate this aspect.

G2. Molecular Backbone Sensing Elements—An Embodiment.

Four types of backbone structure sensing elements (ED, EA, R, Ar) 144, 244, 344, 444 are selected below and illustrated in the embodiment shown in FIG. 5. However, other combinations with more or less different backbone structure sensing elements can be selected, which depend on the needs of specific applications.

The ED or electron donor type of sensing element (or differentiator) 144 preferentially binds only with an electron acceptor (EA) or a highly electron deficient ring system in the analyte 16 to form an electron-exchanged charge complex or a dipole complex. A tetrathiafulvalene (TTF) group is chosen here to illustrate this aspect.

The EA or electron acceptor type of sensing element (or differentiator) 244 preferentially binds only with an electron donor (a highly electron rich ring system) or hydrophilic group in the analyte 16 to form an electron exchanged charge complex, a dipole complex or a hydrogen-bonding complex. A 7,7,8,8-tetra-cyanoquino-dimethane (TCNQ) group is chosen here to illustrate this aspect.

The R type of backbone structure sensing element (or aliphatic group) 344 preferentially binds with the same kind or different kind of aliphatic groups in the analyte 16 to form a molecular complex via van der Waals force interaction. A 2-methyl-butane [—CH₂CH₂CH(CH₃)₂] group is chosen here to illustrate this aspect.

The Ar type of backbone structure sensing element (or aromatic group) 444 preferentially binds with aromatic molecular structures in the analyte 16 to form a molecular complex via van der Waals force interaction. A pyrene (—C₁₆H₉) group is chosen here to illustrate this aspect.

Once the sensor array is prepared by selecting sensing elements, it is important to select the “training set” for pattern recognition. This training set should have all possible analytes that the sensor array could come in contact with. If the sensor is for an expected environment of known vapors, the designing of the training set can be precise. If along with target analytes, the environment also has unknown vapor analytes and interfering analytes, then one should try to represent all possible analytes that might be encountered. The importance of selecting the appropriate training set can be realized by the fact that the quality of the algorithm developed is based on this information.

The dimensions of each sensing unit 10 (sensing element 12 and transducer 14) will affect certain performance characteristics of the sensor array. These dimensions will be so chosen that a certain level of performance (e.g., sensitivity) and operating characteristics (e.g., resonance frequency) could be achieved.

Since it is generally a goal to miniaturize the sensors as much as possible for certain applications without much loss of functionality, sensitivity, or performance, the dimensions of the sensing unit 12 may be in the micrometer range, with the surface 12a having an area in the range of square micrometers. In an ultra-miniaturized form, it may be possible to fabricate the sensing unit 12 even in the submicrometer regime.

Likewise, the thickness of the transducer 14 is generally on the order of one or a few micrometers (“micrometer range”); in the “nanometer range”, the thickness of the transducer is on the order of tens or hundreds of nanometers.

H. Steps to Identify an Unknown Analyte

The following general steps are followed to identify an unknown analyte. In this example, depicted in FIG. 5, there are six CFGS units 142, . . . 642, as described in Table II, and four MSB units 144, . . . 444, as described in Table III.

First, the molecular weight of the analyte is determined using the UMSU 140. In some cases, the analyte may be unambiguously identified without the knowledge of its molecular weight. In most cases, the molecular weight will help determine a particular compound among a group of several as shown below.

Next, a determination is made as to what functional groups are present and what functional groups are absent in the analyte. In some cases, six CFGSUs 142 . . . 642 will be sufficient to indicate the presence or absence of various functional groups on an analyte. A search against the functional groups in Table II will eliminate most of the incorrect choices, leaving a handful of potential candidates. Note that unlike the UMSU 140, where an accurate determination of the molecular weight is essential, it is only required that the CFGSUs 142 . . . 642 indicate the presence or absence of a certain functional group without a high demand on measurement accuracy.

Next, the molecular backbone of the analyte is determined. This process is almost identical to that of determination of the functional groups, with the exception that there are four MSBUs 144 . . . 444. A search against the possible backbones in Table III will result in potential candidates.

A relative high percentage of commonly used chemical compounds may be identified just by going through steps (2) and (3) with the choice of CFGSUs and MSBUs shown in Tables II and III, respectively. However, there may be cases, especially in organic compounds with carbon chains, where it may be necessary to make use of their molecular weights in order to make a determination. Note that by the time this stage is arrived at, there should be only a few potential candidates left in Tables II and III to be identified. By matching the information in the Unit (or Molecular) Mass column in Tables II and III against the molecular weight measurement obtained in the first step above, it should be possible to identify the analyte. In some cases, trying various values of n (or m) may be needed in order to get the best match, i.e., with the least probability of false identification.

The foregoing description has been largely based on using the information received from one or more sensor units 10 to help identify an unknown analyte 16. However, it will also be appreciated that the absence of information from one or more sensor units 10 may also be suitably employed in further aiding the identification of an analyte 16 by eliminating the functional group(s) and/or backbone(s) not sensed.

Below is a brief outline of how the system disclosed herein operates:

The sample is first introduced into the sensor system. It first goes through the sampling and pre-measurement compartment (SPMC) 32 and sample filtering and concentrating compartment (SFCC) 34 (see FIG. 3) for sample preparation. Next, the pretreated single component of analyte is introduced evenly into the three mass sensing units, namely, UMSC 40, CFGSC 42, and MBSC 44, of the molecular sensing compartment (MSC) 36 (see FIG. 4).

The UMSC 40 first measures the mass change and sends its mass information to the data processing unit (DPU) 38. The DPU already has the molar absorption capacity from calibration with known molecules; thus, it will base its determination on the mass change and its known molar absorption capacity to derive the molecular weight.

In the meanwhile, one or more of, say, six CFGSUs 142 . . . 642 within the CFGSC 42 will respond either actively or negatively to this analyte. They will send all their information to the DPU 38. The DPU will refer to the information in Table II, using matching and elimination methods to narrow down what kind of particular functional group(s) the analyte has. This method is usually works well to locate a single functional the analyte has. In the case that the analyte has more than one possible functional group, the DPU 38 will determine the mass difference to further determine what is the functional group by referring to the mass formulations in Table II. In some cases, it may need to combine the information provided from the MBSUs 44 to determine both the particular functional group and its backbone structure.

The MSBUs 144 . . . 444 of MBSC 44 will perform a similar activity to send their either positive or negative responses to the analyte to the DPU 38. The DPU will do the same type of matching and eliminating process in combination with the mass difference to determine what kind of possible backbone structure this analyte has by referring the information provided by Table III. In some cases, the DPU 38 may need to combine the information provided from those CFGSUs 142 . . . 642 of the CFGSC 42 to particularly determine the molecular structure and its functional group(s).

Using all the information from the three compartments 32, 34, 36, through logic analysis and mathematical algorithms, the DPU will finalize what kind of actual molecular structure the particular analyte has.

### EXAMPLES

An Illustration of how to Analyze a Molecular Structure of an Unknown Analyte.

Examples 1-5 below depict how to use Tables II and III to distinguish the chemical functional groups and possible backbone structures.

### Example 1

In this specific example, Analyte 1 is found only adsorbed on CFGSE F-1 and F-5 and on MBSE B-2 and B-4. The adsorption on both F-5 and B-2 indicates that the analyte has one of the following possible functional groups: —OH, —SH, or —NHR. But, adsorption on F-1 instead of both F-1 and F-2 indicates further that the analyte has only a weak base functional group, -φ-NHR, instead of —SH or —OH. The adsorption on B-4 instead of on B-3 confirms the -φ-NHR structure of the molecule with an aromatic ring system.

### Example 2

In this example, Analyte 2 is found only adsorbed on CFGSE F-1 and on MBSE B-4. Adsorption on F-1 (but not both F-1 and F-2) indicates that the analyte has a weak base functional group (—NH₂, —NHR, or —NR₁R₂) connected directly on aromatic ring. But the possibility of being either the —NH₂ or —NHR group is eliminated due to the absence of adsorption on F-5 and B-2. Adsorption on B-4 (but not B-3) confirms that the molecule has an Ar—NR₁R₂ structure and both the R_{1 and R}₂ are very short carbon chain (only 1 or 2 carbon atom(s)) alkyl groups.

### Example 3

In this example, Analyte 3 is found only adsorbed on CFGSE F-3 and F-5 and on MBSE B-2 and B-4. The adsorption on both F-5 and B-2 indicates that the analyte has one of the following possible hydrophilic functional groups: —OH, —SH, or —NHR. Adsorption on B-4 but not on B-3 indicates that the molecule has an aromatic ring system. Adsorption on F-3 rather than on both F-3 and F-4 indicates that the analyte has a weak acid functional group (-φ-OH or -φ-SH), never a base group (e.g. —NHR), connected directly to an aromatic ring. These two functional groups (-φ-OH or -φ-SH) can be distinguished further by their unit mass difference of 16.

### Example 4

In this example, Analyte 4 is found only adsorbed on CFGSE F-1 and on MBSE B-2 and B-4. Adsorption on B-2 (but not on both F-5 and B-2) indicates that the analyte has one of the electron-rich ring systems. The analyte has neither a pyrrole (>NH) ring nor any hydrophilic groups (—OH, —SH, or —NHR), since no (hydrogen-bonding) interaction is observed between F-5 and the analyte. Adsorption on F-1 (but not both F-1 and F-2) indicates that the electron-rich ring system is a weak base, which indicates further this electron-rich weak base is pyridine, not other types of electron-rich ring systems. Finally, adsorption on B-4 confirms further the pyridine-electron rich hetero-aromatic system.

### Example 5

In this example, Analyte 5 is found adsorbed on CFGSE F-1, F-2, and F-6, and only on MBSE B-3. Adsorption on both F-1 and F-2 indicate that the analyte has a strong base functional group, since both strong and weak acid sensing elements can interact with it. No adsorption on F-5, however, indicates that the analyte does not have any hydrophilic groups (—NHR, —NH—NR₁R₂, imidazole or pyrrole), since no (hydrogen-bonding) interaction is observed between F-5 and the analyte. No adsorption on B-2 indicates that the strong base group in the analyte is not an amino pyridine ring. Adsorption on F-6 indicates further that the strong base is a hydrogen bonding-accepting group. The —NR—(C═NH)—NR₁R₂ group seems the only choice. The adsorption on B-3 confirms further the analyte has only an alkyl backbone structure. The molecular formulation and its structure can be finalized once its molecular mass is obtained from UMSU.

For simultaneous identification of all components in the environment, multi-component analysis can be used. Here for this condition, pattern recognition technique plots out responses in n-dimensional hyperspaces, where n is the number of axes corresponding to the number of sensors used in the array. Thus if any unknown interfering analyte is encountered, pattern recognition technique plots out this on a different axis, making discrimination very easy. A diverse set of sensors with strong, selective, and un-correlated responses will more effectively spread different vapors out in feature space, thereby facilitating discrimination.

### INDUSTRIAL APPLICABILITY

The chemical sensor units are expected to find use in micrometer- and nanometer-scale devices and arrays for detecting and analyzing molecules in a variety of environments.

What is claimed is:

1. A sensing unit comprising a chemical sensing element and a transducer operatively associated therewith, wherein said chemical sensing element comprises at least one receptor for receiving a molecule of an analyte.

2. The sensing unit of claim 1 wherein said transducer has two opposed surfaces, with an electrode contact each said opposed surface and wherein said chemical sensing element is either provided on a surface of at least one said electrode or comprises said electrode that is chemically treated.

3. The sensing unit of claim 2 wherein said chemical sensing element interacts with said analyte and generates a physical response, which is read by said transducer and is converted it to an interpretable and quantifiable signal.

4. The sensing unit of claim 3 wherein said transducer comprises a quartz crystal, capable of vibrating in response to an oscillator electrically connected between said electrodes.

5. An array of chemical sensing units, each chemical sensing unit comprising a chemical sensing element and a transducer operatively associated therewith, wherein each said chemical sensing element comprises at least one receptor for receiving a molecule of an analyte.

6. The array of claim 5 wherein said transducer is a quartz crystal micro-balance.

7. The array of claim 5 including three types of chemical differentiators used to identify said analyte: (1) sensing elements for determining either unit mass or molecular weight of said analyte; (2) sensing elements for determining at least one functional group of said analyte; and (3) sensing elements for determining a molecular backbone structure of said analyte.

8. The array of claim 7 wherein said unit mass or said molecular weight is determined by a universal mass-sensing unit.

9. The array of claim 8 wherein said sensing element comprises a universal adsorbent that is capable of adsorbing a broad range of chemicals or biological molecules or species.

10. The array of claim 9 wherein said absorbent is selected from the group consisting of coating materials for gas chromatography or liquid chromatography, activated carbon, silica gel, alumina gel, organometallics, and organic materials.

11. The array of claim 6 wherein said at least one functional group is determined by at least one chemical functional group sensing element.

12. The array of claim 11 wherein said chemical functional group sensing element includes a material selected from the group consisting of strong acids, strong bases, weak acids, weak bases, hydrogen bonding accepting groups, hydrophilic groups, electron donors, and electron acceptors.

13. The array of claim 12 wherein said strong acid binds with any basic group of said analyte to form a proton-exchanged ionic complex.

14. The array of claim 13 wherein said strong acid is selected from the group consisting of —RSO₃H, —CO₂H, —PO(OH)₂, and —PO(OR)OH.

15. The array of claim 12 wherein said strong base binds with any acidic group of said analyte to form a proton-exchanged ionic complex.

16. The array of claim 15 wherein said strong base is selected from the group consisting of alkyl amines, imines, guanidine, hydrazine and its derivatives, azo and its derivatives, and hydroxylamine and its derivatives, alkyl phosphines, alkyl aryl phosphines, and heterocycles containing at least one of nitrogen and phosphorus.

17. The array of claim 12 wherein said weak acid binds only with a strong basic group of said analyte to form a proton-exchanged ionic complex.

18. The array of claim 17 wherein said weak acid is selected from the group consisting of aromatic —OH, aromatic —COOH with a strong electron donating group to deactivate the acid, —B(OH)₂ and its derivatives, and —PO(OH)₂ with a strong electron donating group to deactivate the acid.

19. The array of claim 12 wherein said weak base binds only with a strong acidic group of said analyte to form a proton-exchanged ionic complex.

20. The array of claim 19 wherein said weak base is selected from the group consisting of aromatic amines and deactivated imines with a strong electron withdrawing group to deactivate the imine group, aromatic secondary or tertiary phosphines, and deactivated aromatic phosphines with a strong electron withdrawing group to deactivate the phosphine group.

21. The array of claim 12 wherein said hydrogen bond accepting group binds with a hydrophilic group of said analyte to form a hydrogen bond.

22. The array of claim 21 wherein said hydrogen bond accepting group is selected from the group consisting of —CO—NR₁R₂, —CN, —NO₂, —C(═O)NH₂, —C(═O)NHR, —C(═NH)NH₂, —C(═NR)NH₂, —C(═NH)NHR, —C(═S)NH₂, —C(═O)—SH, —C(═NH)—SH, >C═O, —N═N—, —CF₃, —CCl₃, —CH═O, —SO—, SO₂—, —SO₂OR, —SO₂NR—, —COOR, —C(═O)NR—, and heterocycles containing at least one of nitrogen, oxygen, and sulfur.

23. The array of claim 12 wherein said hydrophilic group binds with a hydrophilic group or hydrogen bond accepting group of said analyte to form a hydrogen bond.

24. The array of claim 23 wherein said hydrophilic group is selected from the group consisting of —OH, —NH₂, SH, —COOH, —C(═O)NH₂, —C(═O)NHR, —C(═S)NH₂, —C(═O)—SH, —C(═NH)NH₂, —C(═NR)NH₂, —C(═NH)NHR, and —C(═NH)—SH.

25. The array of claim 12 wherein said electron donor group binds only with an electron acceptor or a highly electron-deficient ring system of said analyte to form an electron-exchanged charge complex or a dipole complex.

26. The array of claim 25 wherein said electron donor group consists essentially of tetrathiafulvalene, functional groups or structures containing electron-rich hetero atoms or atomic groups or electron-rich unsaturated hydrocarbons.

27. The array of claim 12 wherein said electron acceptor group binds only with an electron donor having a highly electron rich ring system or with a hydrophilic group of said analyte to form an electron exchanged charge complex, a dipole complex or a hydrogen-bonding complex.

28. The array of claim 27 wherein said electron acceptor group is selected from the group consisting of 7,7,8,8-tetra-cyanoquino-dimethane, >C═O, —N═N—, —CN, —NO₂, —CF₃, —CCl₃, —CH═O, —SO—, SO₂—, —SO₂OR, —SO₂NR—, —COOR, —C(═O)NR—, —O—, F, Cl, Br, and I.

29. The array of claim 6 wherein said molecular structure is determined by at least one molecular backbone structure sensing element.

30. The array of claim 29 wherein said backbone structure sensing element includes a material selected from the group consisting of electron donors, electron acceptors, aliphatic groups, aromatic groups, and hydrophobic groups.

31. The array of claim 30 wherein said electron donor group binds only with an electron acceptor (EA) or a highly electron-deficient ring system of said analyte to form an electron-exchanged charge complex or a dipole complex.

32. The array of claim 31 wherein said electron donor group consists essentially of tetrathiafulvalene, functional groups or structures containing electron-rich hetero atoms or atomic groups, or containing electron-rich unsaturated hydrocarbons.

33. The array of claim 31 wherein said electron acceptor group binds only with an electron donor or a highly electron-rich ring system or hydrophilic group of said analyte to form an electron-exchanged charge complex, a dipole complex or a hydrogen-bonding complex.

34. The array of claim 33 wherein said electron acceptor group consists essentially of 7,7,8,8-tetra-cyanoquino-dimethane and functional groups or structures containing highly electron negative heteroatoms or atomic groups.

35. The array of claim 30 wherein said aliphatic group binds with the same or different kind of aliphatic groups in said analyte to form a molecular complex via van der Waals force interaction.

36. The array of claim 35 wherein said aliphatic group consists essentially of saturated hydrocarbons, unsaturated hydrocarbons, and heteroatom-substituted hydrocarbons.

37. The array of claim 30 wherein said aromatic group binds with an aromatic molecular structure in said analyte to form a molecular complex via van der Waals force interaction.

38. The array of claim 37 wherein said aromatic group consists essentially of single ring aromatic systems, multi-ring aromatic systems, aromatic heterocycles, and heteroatom-substituted hydrocarbons.

39. The array of claim 30 wherein said hydrophobic group binds with the same or different kinds of hydrophobic groups to form a molecular complex via van der Waals force interaction.

40. The array of claim 39 wherein said hydrophobic group is selected from the group consisting of aliphatic groups, aromatic groups, ethers, thio ethers, esters, thiol esters, and tertiary amines.

41. A chemical sensor system comprising an array of chemical sensing units, each chemical sensing unit comprising a chemical sensing element and a transducer, each chemical sensing element comprising at least one receptor for receiving a molecule of an analyte, said chemical sensor system capable of identifying at least one molecule of an analyte.

42. The sensor system of claim 41 comprising, in sequence, a sampling and pre-measurement compartment, a sample filtration and concentration compartment, at least one molecule sensing compartment, and a data processing unit.

43. The sensor system of claim 42 wherein said at least one molecule sensing compartment comprises three sub-compartments: (1) a universal mass sensing sub-compartment; (2) a chemical functional group sensing sub-compartment, and (3) a molecular backbone structure sensing sub-compartment.

44. The sensor system of claim 45 wherein said universal mass sensing sub-compartment comprises a pair of mass sensing units: (1) a universal mass sensing unit and (2) a reference mass sensing unit, wherein the reference mass sensing unit is a special mass sensing unit without any sensing element on top of its transducer.

45. The sensor system of claim 43 wherein said chemical functional group sensing sub-compartment comprises an array of chemical functional group sensing units.

46. The sensor system of claim 45 wherein each said chemical functional group sensing unit is constructed from a set of functional group sensing elements and an equal number of individual transducers, wherein said functional group sensing elements each include a material independently selected from the group consisting of (1) strong acids, which bind with any basic group of said analyte to form a proton-exchanged ionic complex, (2) strong bases, which bind with any acidic group of said analyte to form a proton-exchanged ionic complex, (3) weak acids, which bind only with a strong basic group of said analyte to form a proton-exchanged ionic complex, (4) weak bases, which bind only with a strong acidic group of said analyte to form a proton-exchanged ionic complex, (5) hydrogen bonding accepting groups, which bind with a hydrophilic group of said analyte to form a hydrogen bond, (6) hydrophilic groups, which bind with a hydrophilic group or hydrogen bond accepting group of said analyte to form a hydrogen bond, (7) electron donors, which bind only with an electron acceptor or a highly electron-deficient ring system of said analyte to form an electron-exchanged charge complex or a dipole complex, and (8) electron acceptors, which bind only with an electron donor having a highly electron rich ring system or with a hydrophilic group of said analyte to form an electron exchanged charge complex, a dipole complex or a hydrogen-bonding complex.

47. The sensor system of claim 43 wherein said molecular backbone structure sensing sub-compartment comprises an array of molecular backbone structure sensing units.

48. The sensor system of claim 47 wherein each said molecular backbone structure sensing unit is constructed from backbone structure sensing elements and an equal number of individual transducers, wherein said backbone structure sensing elements each include a material independently selected from the group consisting of (1) electron donors, which bind only with an electron acceptor (EA) or a highly electron-deficient ring system of said analyte to form an electron-exchanged charge complex or a dipole complex, (2) electron acceptors, which bind only with an electron donor or a highly electron-rich ring system or hydrophilic group of said analyte to form an electron-exchanged charge complex, a dipole complex or a hydrogen-bonding complex, (3) aliphatic groups, which bind with the same or different kind of aliphatic groups in said analyte to form a molecular complex via van der Waals force interaction, (4) aromatic groups, which bind with an aromatic molecular structure in said analyte to form a molecular complex via van der Waals force interaction, and (5) hydrophobic groups, which bind with the same or different kinds of hydrophobic groups to form a molecular complex via van der Waals force interaction.

49. A method of detecting and identifying an analyte comprising at least one molecular species, comprising:
providing a chemical sensor system comprising, in sequence, a sampling and pre-measurement compartment, a sample filtering and concentration compartment, at least one molecule sensing compartment, and a data processing unit;
introducing said analyte into each said compartment in turn;
sensing a mass of said at least one molecular species;
sensing at least one chemical functional group on said at least one molecular species;
sensing a backbone structure of said at least one molecular species; and
analyzing information relating to said mass, said at least one functional group, and said backbone structure to provide an identity of said at least one molecular species of the analyte.

50. The method of claim 49 wherein said at least one molecule sensing compartment comprises three sub-compartments: (1) a universal mass sensing sub-compartment; (2) a chemical functional group sensing sub-compartment, and (3) a molecular backbone structure sensing sub-compartment.

51. The method of claim 50 wherein said universal mass sensing sub-compartment comprises a pair of mass sensing units: (1) a universal mass sensing unit and (2) a reference mass sensing unit, wherein the reference mass sensing unit is a special mass sensing unit without any sensing element on top of its transducer.

52. The method of claim 51 wherein said universal mass sensing unit has the capacity to absorb the same molar quantities of molecules each time so as to permit determining said molecular weight of said analyte.

53. The method of claim 51 wherein said reference mass sensing unit is configured to track inherent drift of said transducer due to environmental conditions and to make any suitable adjustments to compensate for said drift.

54. The method of claim 50 wherein said chemical functional group sensing sub-compartment comprises an array of chemical functional group sensing units.

55. The method of claim 54 wherein each said chemical functional group sensing unit is constructed from a set of functional group sensing elements and an equal number of individual transducers, wherein said functional group sensing elements each include a material independently selected from the group consisting of (1) strong acids, which bind with any basic group of said analyte to form a proton-exchanged ionic complex, (2) strong bases, which bind with any acidic group of said analyte to form a proton-exchanged ionic complex, (3) weak acids, which bind only with a strong basic group of said analyte to form a proton-exchanged ionic complex, (4) weak bases, which bind only with a strong acidic group of said analyte to form a proton-exchanged ionic complex, (5) hydrogen bonding accepting groups, which bind with a hydrophilic group of said analyte to form a hydrogen bond, (6) hydrophilic groups, which bind with a hydrophilic group or hydrogen bond accepting group of said analyte to form a hydrogen bond, (7) electron donors, which bind only with an electron acceptor or a highly electron-deficient ring system of said analyte to form an electron-exchanged charge complex or a dipole complex, and (8) electron acceptors, which bind only with an electron donor having a highly electron rich ring system or with a hydrophilic group of said analyte to form an electron exchanged charge complex, a dipole complex or a hydrogen-bonding complex.

56. The method of claim 55 wherein said chemical functional group sensing unit permits determination of what functional groups are present and what functional groups are absent.

57. The method of claim 50 wherein said molecular backbone structure sensing sub-compartment comprises an array of molecular backbone structure sensing units.

58. The method of claim 57 wherein each said molecular backbone structure sensing unit is constructed from backbone structure sensing elements and an equal number of individual transducers, wherein said backbone structure sensing elements each include a material independently selected from the group consisting of (1) electron donors, which bind only with an electron acceptor (EA) or a highly electron-deficient ring system of said analyte to form an electron-exchanged charge complex or a dipole complex, (2) electron acceptors, which bind only with an electron donor or a highly electron-rich ring system or hydrophilic group of said analyte to form an electron-exchanged charge complex, a dipole complex or a hydrogen-bonding complex, (3) aliphatic groups, which bind with the same or different kind of aliphatic groups in said analyte to form a molecular complex via van der Waals force interaction, (4) aromatic groups, which bind with an aromatic molecular structure in said analyte to form a molecular complex via van der Waals force interaction, and (5) hydrophobic groups, which bind with the same or different kinds of hydrophobic groups to form a molecular complex via van der Waals force interaction.

59. The method of claim 58 wherein said molecular backbone structure sensing unit permits determination of what backbone structures are present and what backbone structures are absent.
